(19) United States
(12) Patent Application Publication
(10) Pub. No.: US 2007/0015697 A1
(43) Pub. Date: Jan. 18, 2007

(54) Enhanced ocular neuroprotection and neurostimulation

(76) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(21) Appl. No.: 11/263,737

(22) Filed: Nov. 1, 2005

Related U.S. Application Data
(63) Continuation-in-part of application No. 11/183,355, filed on Jul. 18, 2005.

(51) Int. Cl. A61K 38/18; A61K 38/13; A61K 31/7048; A61K 31/4745

(52) U.S. Cl. 514/12; 514/11; 514/28; 514/29; 514/291

(57) ABSTRACT

An ocular method comprising non-systemic localized ocular administration of a pharmaceutically acceptable formulation and effective concentration of at least one neurostimulatory and/or neuroprotective macrolide or other agent for a duration sufficient to enhance viability, confer protection, reduce degeneration of retinal neural cells. The method is used in a patent having or at risk for developing glaucoma, retinitis pigmentosa, or another ocular neuroassociated disease.

This application is a Continuation-In-Part of U.S. patent application Ser. No. 11/183,355 filed Jul. 18, 2005, which is expressly incorporated by reference herein in its entirety.

### FIELD OF THE INVENTION

A method to enhance ocular neuroprotection and/or neurostimulation to reduce neurodegenerative changes that may be associated with glaucoma or other ocular diseases.

### BACKGROUND

Methods and compositions that enhance a patient's condition prophylactically or therapeutically, or after ocular surgery are desirable.

### SUMMARY OF THE INVENTION

One embodiment of the invention is a composition comprising at least one neuro-stimulatory factor in a pharmaceutically effective concentration and formulation for non-systemic localized ocular administration and effect. The composition contains macrolides or may further be modified to contain one or more macrolides if not already present. It may be formulated with excipients for topical ocular administration, administration in an ocular device or delayed release matrix, administration by subconjunctival or intraocular injection, etc. It may be contained in an intraocular implant, an intraocular lens, or a contact lens. The neuroprotective or neurostimulatory factor may be a macrolide, which may be cyclosporin A, tacrolimus, sirolimus, everolimus, pimocrolous, or others; macrolide analog; neurotrophin; and/or a neuropoietic factor. In some embodiments, one or more other agents may also be included, for example, an antioxidant, steroid, non-steroidal anti-inflammatory drug, antibiotic, anti-proliferative agent, anti-cell migration agent, anti-prostaglandin, anti-angiogenic agent, vitamin, mineral, growth factor, or cytokine.

Another embodiment is an ocular method comprising administering to a patient after ocular surgery a composition comprising at least one neuro-stimulatory factor, which also encompasses a macrolide or macrolide analog with neuro-stimulatory activity, in a pharmaceutically effective concentration and formulation for non-systemic localized ocular administration. The composition may be ocularly administered topically, subconjunctivally, intraocularly, by implantation in a device or a lens, or from a contact lens. The composition may be administered to the patient after corneal surgery such as laser-assisted in situ keratomileusis (LASIK), photorefractive keratectomy (PRK), total corneal transplant, or partial corneal transplant.

Another embodiment is an ocular method whereby a macrolide or macrolide analog is administered to a post-ocular surgery patient to reduce or minimize ocular scarring. The macrolide may be present as a component in a composition administered to provide a neuroprotective and/or neurostimulatory effect. Alternatively, the macrolide may be administered to reduce or minimize scarring following any type of ocular surgery, including but not limited to glaucoma surgery, retinal detachment repair surgery, and corneal surgery.

Another embodiment is a method for administering a macrolide, macrolide analog, neurotrophin, and/or neuropoietic agent prophylactically to patients having or at risk for developing an ocular neurologic or neurosensory disease, or therapeutically to patients with an ocular neurologic or neurosensory disease, either alone or in conjunction with other therapy. Glaucoma is a non-limiting example of an ocular disease with a neuroassociated component. Retinitis pigmentosa is a non-limiting example of an ocular disease with a neurosensory component

These and other embodiments of the invention will be further appreciated in view of the following detailed description.

### DETAILED DESCRIPTION

One embodiment is a method to enhance patient recovery after ocular surgery or other trauma by enhancing corneal sensation, ocular nerve regeneration, and/or re-enervation. The method at least partially restores the loss of corneal sensation that occurs following corneal procedures during which nerves are severed. The method also reduces or minimizes post-surgical scarring that could lead to corneal opacification, reduced vision, and/or other complications in compositions with a macrolide or macrolide analog component. For example, it could be used to reduce or minimize scarring of the conjunctiva that occurs after glaucoma surgery, or scarring that may lead to proliferative vitreal retinopathy (PVR) after retinal detachment repair surgery, or scarring that occurs after corneal surgery. While not being bound by a specific theory, a method to reduce or minimize post ocular-surgery scarring may enhance ocular sensation, nerve regeneration, and/or re-enervation, possibly by minimizing scar tissue that may impair nerve growth, nerve cell connections, etc. The method thus leads to enhanced recovery following ocular surgery.

One embodiment provides localized ocular administration of macrolides and/or macrolide analogs, either alone or in combination with other neuro-stimulatory agents such as neurotrophins, neuropoietins, etc. The macrolides and/or macrolide analogs may or may not have neuro-stimulatory activity.

“Corneal anesthesia” is an unwanted consequence in some patients who have undergone an ocular surgical procedure. Such procedures include laser-assisted in situ keratomileusis (LASIK), photorefractive keratectomy (PRK), and corneal transplant (total or partial). In these types of procedures, the surgeon creates a micro-thin flap in the cornea and stroma to access the cornea. The stromal corneal flap may be created using a femtosecond computer-guided laser, or a hand-held microkeratome with an oscillating metal blade. The flap is then folded open to provide access to the cornea for the procedure, after which the flap is then return to its original position where it seals without stitches. The flap promotes post-surgical healing, patient comfort, and improved vision. If the flap is not of the proper thickness (e.g., too thick, too thin, or irregular), the patient's healing and quality of vision may be compromised.

In creating the flap, the nerves that enervate the surface of the cornea are necessarily cut. One study reported that the number of sub-basal and stromal nerve fiber bundles in the corneal flap decreased 90% immediately following the surgery. Although the sub-basal nerve fiber bundles gradually returned, their number remained less than half of the pre-surgical number. The loss of corneal sensation caused by a decrease in the number of enervating nerves, and/or their function, may last up to about six months after the original procedure. Diabetic patients are particularly prone to decreased corneal nerve function, yet are a group of patients in frequent need of corneal transplants.

After corneal surgery patients may experience problems relating to the loss of ocular sensitivity or sensation. For example, decreased ocular nerve function makes the cornea prone to trauma, which in turn can lead to infection. It reduces the usual blink mechanism that is required to keep the corneal surface moist, leading to drying and sloughing of the corneal epithelium. This, in turn, causes cloudiness of the flap, prones the flap to infection by enteral pathogens because of loss of barrier, and reduces vision.

One embodiment of the invention locally administers one or more agents that enhance corneal sensation, possibly by nerve regeneration and/or enervation. In one embodiment, one or a combination of macrolides, including macrolide analogues, is administered, the macrolide and/or analogue having neuro-stimulatory activity. In another embodiment, one or a combination of macrolides is administered with one or more agent(s) that enhance corneal nerve stimulation. Such neuro-stimulatory agents may increase nerve cell quantity, functional quality, or combinations of these. One skilled in the art will appreciate that enhancement refers to any qualitative and/or quantitative improvement in corneal sensation and/or ocular neurological function following surgery regardless of degree.

One embodiment of the invention is a composition containing a neuro-stimulatory or neuroprotective macrolide, macrolide analog, neurotrophin, and/or neuropoietic factor that may be administered prophylactically for patients having or at risk for developing glaucoma, retinitis pigmentosa, or other neurosensory or neurodegenerative disease, or may be administered to patients with glaucoma or retinitis pigmentosa, either alone or in conjunction with other therapy.

Glaucoma is a general term for several types of a painless ocular condition that, left untreated, can result in partial or complete vision loss. It is characterized by elevated intraocular pressure, considered by one skilled in the art as a pressure greater than about 21.5 mm Hg. The higher the intraocular pressure, the greater the likelihood of optic nerve damage and visual field loss. In glaucoma monitoring or therapy, the neurodegenerative component, such as protection of retinal ganglion cells (RGC), should be considered in addition to therapy for increased intraocular pressure.

Known risk factors for glaucoma include age (elevated risk for individuals over age 60), race (elevated risk for African Americans over age 40), a family history of glaucoma, individuals with diabetes, severe nearsightedness, long-term corticosteroid use, previous eye injury, and/or increased intraocular pressure. One risk factor may suffice for prophylactic administration of a neuroprotective and/or neurostimulatory agent as described herein, and risk factors may alter over time, as known to one skilled in the art.

In monitoring, diagnosing, and/or treating patients with glaucoma, attainment of decreased intraocular pressure is a necessary but insufficient goal. This is because a component of glaucoma is neurologic damage to the optic nerve and ganglion cell death, so that its neurodegenerative aspects must be considered. Even patients with normal intraocular pressure may develop glaucoma-like changes. Further, retinal ganglion cells may be more sensitive to increased intraocular pressure, whereas other ocular cells may be better able to withstand increased intraocular pressure.

Retinitis pigmentosa is a general term that encompasses a disparate group of disorders of rods and cones. Because retinitis pigmentosa affects these retinal sensory structures, prophylactic or therapeutic administration of neuroprotective or neurostimulatory agents may reduce decreased visual field and other adverse effects.

In one embodiment, a patient is prophylactically or therapeutically administered a neurostimulatory and/or neuroprotective macrolide, macrolide analog, neurotrophin, and/or neuropoietic agent. The inventive method may prevent or delay an increase in intraocular pressure, may reduce associated nerve loss, may confer protection on retinal sensory cells, etc. Administration may be by any route. One example is topical application, with the neurostimulatory and/or neuroprotective agent(s) administered in a formulation of eye drops, cream, ointment, gel, salve, etc. Another example is intraocular injection with the neurostimulatory and/or neuroprotective agent administered subconjunctivally, intravitreally, retrobulbarly, within the crystalline lens via piercing the lens capsule as described in co-pending U.S. patent application Ser. No. 11/103,283 which is expressly incorporated by reference herein, etc. Another example provides the neurostimulatory and/or neuroprotective agent to the eye on or in a formulation such as a liposome, microsphere, microcapsule, biocompatible matrix, gel, polymer, nanoparticle, nanocapsule, etc. Another example provides the neurostimulatory and/or neuroprotective agent on or in a device such as a device for transscleral delivery as described in co-pending U.S. patent application Ser. No. 11/105,756, or another intraocular device using, for example, iontophoresis or another type of release mechanism (controlled or not controlled), as known by one skilled in the art. Another example provides the neurostimulatory and/or neuroprotective agent in conjunction with gene therapy, as known by one skilled in the art.

Ganglion cells in the retina (RGC) that have been damaged (e.g., by elevated intraocular pressure) undergo apoptosis, also referred to as programmed cell death. The macrolide tacrolimus, systemically administered, conferred neuroprotection on RGC by interfering with apoptotic mechanisms, as disclosed in Freeman and Grosskreutz Investigative Ophthalmology & Visual Science:41, 1111 (2000), which is expressly incorporated by reference herein in its entirety. As a result of programmed cell death, RGC release compounds whose presence and/or concentration may result in toxicity, remove desirable agent and/or alter cell signaling; these compounds include cytokines, the excitatory neurotransmitter glutamate, Ca²⁺ binding proteins, FK 506 (tacrolimus) binding proteins, and others. Thus, ocular administration of a neurostimulatory and/or neuroprotective macrolide, macrolide analog, neurotrophin, and/or neuropoietic factor may reduce or inhibit subsequent effects of the released cytokines, glutamate, etc. that are part of the neurodegenerative processes associated with glaucoma and/or retinitis pigmentosa. For example, macrolides tacrolimus (FK 506) and cyclosporin A are potent immunosuppresants that inhibit T-cell activation by interfering with signal transduction. In vitro, tacrolimus binds to and inhibits the activity of the immunophilin FK 506-binding protein (FKBP), an isomerase that functions in signal transduction and cell communication. Reducing apoptotic mechanisms would reduce such processes, and thus protect or delay neurosensory impairment or neurodegenerative damage.

Such administration of macrolides may be alone or may be in conjunction with other agents used to reduce intraocular pressure in patients with elevated intraocular pressure due to ocular hypertension or open-angle glaucoma. For example, administration may be included with a current drug regimen, or at different intervals than a current regimen, or for a set duration, etc; all these are examples of administration in conjunction with other agent. Examples of known drugs include, but are not limited to, Diamox® (acetazolamide(N-(5-sulfamoyl-1,3,4-thiadiazol-2-yl)acetamide), an inhibitor of carbonic anhydrase, Wyeth, Madison N.J.); Timoptic® (timolol maleate ophthalmic solution, Merck, Whitehouse Station N.J.), Xalatan® (latanaprost ophthalmic solution, Pfizer, Groton Calif.); Copaxone® (Teva Pharmaceuticals, Petah Tiqva, Israel); Memantine (Allergan, Irvine Calif.); Alphagan® P (brimonidine tartrate ophthalmic solution; Allergan); and others known to one skilled in the art. In this embodiment, the inventive method may use macrolides to potentiate the action of current treatments. For example, acetazolamide may assist in normal polarization of cell membranes, so the effect of local ocular treament with acetazolamide and a neuroprotective macrolide or agent, is reduced apoptic effects and normalized polarization of sensory retinal ganglion cells. The dual action may be additive or synergistic.

Macrolides encompassed by the invention are those known by one skilled in the art, as well as analogs and derivatives. These are disclosed in, for example, co-pending U.S. patent application Ser. Nos. 10/667,161 and 10/752,124. Macrolides and their analogues that may be administered include the following.

Cyclosporin A (cyclosporine, topical formulation Arrestase®, Allergan Inc.) is a cyclic peptide produced by Trichoderma polysporum. It is available commercially, for example, from Sigma-Aldrich (St. Louis Mo.). It is an immunosuppressant and acts in a particular subset of T lymphocytes, the helper T cells. Cyclosporin A exerts an immunosuppressant effect by inhibiting production of the cytokine interleukin 2. Each of Cyclosporin A and tacrolimus, another immunosuppressant, produces significant renal and hepatic toxicity when each is administered systemically; because of this toxicity, they are not administered together. The use of Cyclosporin A as a specific medicament for treatment of ocular disease with reduced toxicity is described in co-pending U.S. patent application Ser. No. 10/289,772.

Tacrolimus (Prograf®, previously known as FK506), a macrolide immunosuppressant produced by Streptomyces tsukubaensis, is a tricyclo hydrophobic compound that is practically insoluble in water, but is freely soluble in ethanol and is very soluble in methanol and chloroform. It is available under prescription as either capsules for oral administration or as a sterile solution for intravenous administration. The solution contains the equivalent of 5 mg anhydrous tacrolimus in 1 ml of polyoxyl 60 hydrogenated castor oil (HCO-60), 200 mg, and dehydrated alcohol (USP, 80.0%^{v/v}), and must be diluted with a solution of 0.9% NaCl or 5% dextrose before use.

Sirolimus, also known as rapamycin, RAPA, and Rapamune®, is a triene macrolide antibiotic derived from Streptomyces hydroscopicus and originally developed as an antifungal agent. Subsequently, it has shown anti-inflammatory, anti-tumor, and immunosuppressive properties. Pimecrolimus, also known as ascomycin, Immunomycin, and FR-900520, is an ethyl analog of tacrolimus and has strong immunosuppressant properties. It inhibits Th1 and Th2 cytokines, and preferentially inhibits activation of mast cells, and is used to treat contact dermatitis and other dermatological conditions. Sirolimus and pimecrolimus are commercially available, e.g., A.G. Scientific, Inc. (San Diego, Calif.).

Regarding its immunosuppressive potential, sirolimus has some synergetic effect with Cyclosporin A. It has been reported that sirolimus has a different mode of action compared to Cyclosporin A and tacrolimus. All three agents are immunosuppressants which affect the action of immune cell modulators (cytokines), but do not affect the immune cells themselves. However, while all three agents affect immune cell modulators, they do so differently: Cyclosporin A and tacrolimus prevent synthesis of cytokine messengers, specifically interleukin-2, while sirolimus acts on cytokine that has already been synthesized, preventing it from reaching immune cells.

Sirolimus inhibits inflammation by acting on both T-lymphocytes and dendritic cells. The latter are the first cells to recognize antigens. Sirolimus blocks the growth of dendritic cells and a number of other cells, such as tumors and endothelial cells, which are activated by the tumor cell releasing vascular endothelial growth factor (VEGF). VEGF is a central regulator of angiogenesis (formation of new blood vessels from pre-existing vessels) and vasculogenesis (development of embryonic vasculature through an influence on endothelial cell differentiation and organization). Diseases that are characterized by abnormal angiogenesis and vasculogenesis, such as some cancers and some ocular diseases, may show abnormal production of VEGF. Thus, control of VEGF function may be one means to control or treat these diseases. Sirolimus has also been used in the prevention of smooth muscle hyperplasia after coronary stent surgery. The use of sirolimus and ascomycin as specific medicaments for treatment of ocular disease has been disclosed in co-pending U.S. patent application Ser. No. 10/631,143.

Everolimus, also known as RAD-001, SCZ RAD, Certican™ (Novartis, Basel Switzerland), is an analog of sirolimus but is a new and distinct chemical entity. It is an oral immunosuppressant that inhibits growth factor-induced cell proliferation and thus reduces acute organ rejection and vasculopathy, the proliferation of smooth muscle cells in the innermost wall of grafts that restricts blood supply.

It will be appreciated that the invention encompasses the use of macrolides in addition to those previously described. These include, for example, the known antibiotics erythromycin and its derivatives such as azithromycin and clarithromycin, lincomycin, dirithromycin, josamycin, spiramycin, diacetyl-midecamycin, troleandomycin, tylosin, and roxithromycin, and other macrolides such as biolimus, ABT-578 (methylrapamycin); macrolide derivatives such as temsirolimus (CCI-779, Wyeth) and AP23573 (Ariad) (both rapamycin derivatives). The invention also includes new macrolide antibiotic scaffolds and derivatives in development, including but not limited to the ketolides ABT-773 and telithromycin as described by Schonfeld and Kirst (Eds.) in Macrolide Antibiotics, Birkhauser, Basel Switzerland (2002); macrolides derived from leucomycins, as described in U.S. Pat. Nos. 6,436,906; 6,440,942; and 6,462,026 assigned to Enanta Pharmaceuticals (Watertown Mass.); and lincosamides.

Any of the above-described macrolides may be used in the invention. In one embodiment, the total macrolide concentration ranges from less than 1 ng/ml to about 10 mg/ml. In another embodiment, the total macrolide concentration ranges from about 1 ng/ml to about 1 mg/ml. In another embodiment, the total macrolide concentration is below 5 mg/ml.

Specific macrolide analogues accelerate nerve regeneration and functional recovery, as disclosed in Revill et al., J. Pharmacol. Exp. Therap. (2002) 302; 1278, which is expressly incorporated by reference herein in its entirety. For example, genetically engineered 13- and 15-desmethoxy analogs of ascomycin, examples of macrolide analogs, that contain hydrogen, methyl, or ethyl instead of methoxy at either the 13-, the 15-, or both the 13- and 15-positions enhanced neurite outgrowth in cultured SH-SY5Y neuroblastoma cells at concentrations of 1 mg/kg/day and 5 mg/kg/day, with nerve growth factor (NGF) at a concentration of 10 ng/ml. The ascomycin analog 13-desmethoxy-13-methyl-18 hydroxy (13-Me-18-OH), at concentrations of 1 mg/kg/day and 5 mg/kg/day, was demonstrated to accelerate nerve regeneration and lead to full functional recovery (walking) in a rat sciatic nerve crush model.

The combination of a macrolide and another neurostimulatory or neuroprotective factor(s) such as neurotrophins or neuropoietins is used in one embodiment.

Neurotrophins are a family of polypeptides that enhance survival of nervous tissue by maintenance, growth, differentiation, etc. They stimulate the growth of sympathetic and sensory nerve cells in both the central and peripheral nervous system. All neurotrophins have six conserved cysteine residues and share a 55% amino acid sequence identity. Some are in a pro-neurotrophin form and are cleaved to produce a mature form. Examples of neurotrophins include nerve growth factor-β (NGFβ), brain-derived neurotrophic factor (BDNF), neurotrophin 3 (NT-3), neurotrophin 4 (NT-4), neurotrophin 6 (NT-6). These are available commercially, for example, from Sigma-Aldrich (St. Louis Mo.); Axxora (San Diego Calif.) mouse 2.5S and 7S components NGFβ, human recombinant β-NGF and pro-β-NGF.

Different neuron types require different neurotrophins, depending upon their receptor expression. All neurotrophins are capable of binding to p75 neurotrophin growth factor receptors, which are low affinity receptors. Specific neurotrophins and mature neurotrophins bind to different tyrosine kinase (trk) receptors, which are higher affinity receptors than p75 receptors. Tyrosine kinase receptors include types A (trkA), B (trkB), and C (trkC).

NGFβ is a specific ligand for the trkA receptor and signals through trkA. It also signals through the low affinity p75 receptors. NGFβ is a secreted protein that helps to develop and maintain the sympathetic nervous system, affecting sensory, pain, and sympathetic targets. It is required for survival of small, peptide-expressing neurons that express the trkA receptor and that project into the superficial laminae of the dorsal horn (i.e., putative nociceptive neurons).

BDNF signals through trkB, in addition to the low affinity p75 receptors. It is Ca²⁺ dependent and may control synaptic transmission and long term synaptic plasticity, affecting sensory and motor targets. It enhances survival and differentiation of several classes of neurons in vitro, including neural crest and placode-derived sensory neurons, dopaminergic neurons in the substantia nigra, basal forebrain cholinergic neurons, hippocampal neurons, and retinal ganglial cells. BDNF is expressed within peripheral ganglia and is not restricted to neuronal target fields, so that it may have paracrine or autocrine actions on neurons as well as non-neuronal cells.

Neurotrophin-3 (NT-3) is part of the family of neurotrophic factors that control survival and differentiation of mammalian neurons. NT-3 is closely related to NGFβ and BDNF. The mature NT-3 peptide is identical in all mammals examined including human, pig, rat and mouse. NT-3 preferentially signals through trkC, over trkA and trkB receptors, and also utilizes the low affinity p75 receptors. It functions at the neuromuscular junction, affecting large sensory and motor targets and regulating neurotransmitter release at neuromuscular synapses. It may be involved in maintenance of the adult nervous system, and affect development of neurons in the embryo when it is expressed in human placenta.

Neurotrophin 4 (NT-4, synonymous with NT-5) belongs to the NGF-β family and is a survival factor for peripheral sensory sympathetic neurons. NT-4 levels are highest in the prostate, with lower levels in thymus, placenta, and skeletal muscle. NT-4 is also expressed in embryonic and adult tissues. It signals through trkB in addition to low affinity p75 receptors, affecting sympathetic, sensory, and motor targets. Neurotrophin-6 has also been reported.

Ciliary neurotrophic factor (CNTF) is expressed in glial cells within the central and peripheral nervous systems. It stimulates gene expression, cell survival, or differentiation in a variety of neuronal cell types such as sensory, sympathetic, ciliary, and motor neurons. CNTF itself lacks a classical signal peptide sequence of a secreted protein, but is thought to convey its cytoprotective effects after release from adult glial cells by some mechanism induced by injury. In addition to its neuronal actions, CNTF also acts on non-neuronal cells such as glia, hepatocytes, skeletal muscle, embryonic stem cells, and bone marrow stromal cells.

Glial cell derived neurotrophic factor (GDNF) is a 20 kD glycosylated polypeptide that exists as a homodimer. It stimulates the growth of dopaminergic neurons and autonomic motor neurons.

Neuropoietic factors may be used in addition to, or in place of, neurotrophic factors. Neuropoietic factors regulate the properties of cells both in the peripheral and central nervous systems, and both during development and in the mature nervous system. They regulate neuronal phenotype (neurotransmitter) and differentiation of neuronal precursor cells in peripheral and spinal cord neurons. They also regulate cell survival, and development of astrocytes and oligodendrocytes. Neuropoietic factors are also trauma factors in rescuing sensory and motor neurons from axotomy-induced cell death. They show temporal and spatial specific expression patterns, and have specific roles in neural development and repair.

Neuropoietic factors include some cytokines, different from cytokines associated with apoptosis-induced neurodegenerative processes, and hematopoietic factors that fulfill criteria for demonstrating a role in neuronal differentiation and survival. They include leukemia inhibitory factor (LIF), oncostatin M, growth-promoting activity, and cardiotrophin 1. All of these factors activate a subfamily of class I cytokine receptors, the interleukin-6 receptor family.

Any of the above-described neurotrophins and/or neuropoietic factors may be used in the invention. In one embodiment, the total concentration of neurotrophins and/or neuropoietic factors ranges from about 1 pM to about 100 pM. In another embodiment, the total concentration of neurotrophins and/or neuropoietic factors ranges from about 0.01 nM to about 1 M. In another embodiment, the total concentration of neurotrophins and/or neuropoietic factors is below 1 nM. The neurotrophin(s) and/or neuropoietic factor(s) may be used singly or in combination.

The addition of a macrolide, macrolide analog, neurotrophin and/or a neuropoietic factor, alone or in combination, in an ocular formulation, provides beneficial results in enhancing corneal sensation, nerve regeneration, protection, and/or re-enervation. In embodiments where a macrolide is present, the composition also reduces post ocular surgical scarring, and provides anti-inflammatory and anti-infective properties. It will be appreciated that various embodiments are contemplated. As one example, a macrolide or macrolide analog, with or without neurostimulatory activity, may be used without a neurotrophin or neuropoietic factor. As another example, a neurotrophin or neuropoietic factor or any other neuro-stimulatory factor or factors may be used alone. As another example, other agents may be included in the composition. Examples of these agents include, but are not limited to, steroids, non-steroidal anti-inflammatory agents (NSAIDS), antibiotics, antioxidants, anti-proliferative, anti-cell migration, and/or anti-angiogenic agents.

Steroids for ocular administration include, but are not limited to, triamcinolone (Aristocort®; Kenalog®), betamethasone (Celestone®), budesonide, cortisone, dexamethasone (Decadron-LA®; Decadron® phosphate; Maxidex® and Tobradex® (Alcon)), hydrocortisone, methylprednisolone (Depo-Medrol®, Solu-Medrol®), prednisolone (prednisolone acetate, e.g., Pred Forte® (Allergan); Econopred and Econopred Plus® (Alcon); AK-Tate® (Akorn); Pred Mild® (Allergan); prednisone sodium phosphate (Inflamase Mild and Inflamase Forte® (Ciba); Metreton® (Schering); AK-Pred® (Akorn)), fluorometholone (fluorometholone acetate (Flarex® (Alcon); Eflone®), fluorometholone alcohol (FML® and FML-Mild®, (Allergan); Fluor OP®)), rimexolone (Vexol® (Alcon)), medrysone alcohol (HMS® (Allergan)); lotoprednol etabonate (Lotemax®) and Alrex® (Bausch & Lomb), 11-desoxcortisol, and anacortave acetate (Alcon)).

Antibiotics include, but are not limited to, doxycycline (4-(dimethylamino)-1,4,4a,5,5a,6,11,12a-octahydro-3,5,10,12,12a-pentahydroxy-6-methyl-1,11-dioxo-2-naphthacenecarboxamide monohydrate, C₂₂H₂₄N₂O₈.H₂O), aminoglycosides (e.g., streptomycin, amikacin, gentamicin, tobramycin), cephalosporins (e.g., beta lactams including penicillin), tetracyclines, acyclorvir, amantadine, polymyxin B, amphtotericin B, amoxicillin, ampicillin, atovaquone, azithromycin, azithromycin, bacitracin, cefazolin, cefepime, cefotaxime, cefotetan, cefpodoxime, ceftazidime, ceftizoxime, ceftriaxone, cefuroxime, cephalexin, chloramphenicol, clotimazole, ciprofloxacin, clarithromycin, clindamycin, dapsone, dicloxacillin, erythromycin, fluconazole, foscarnet, ganciclovir, gatifloxacin, griseofulvin, isoniazid, itraconazole, ketoconazole, metronidazole, nafcillin, neomycin, nitrofurantoin, nystatin, pentamidine, rifampin, rifamycin, valacyclovir, vancomycin, etc.

Anti-proliferative agents include, but are not limited to, carboplatin, 5-fluorouracil (5-FU), thiotepa, etoposide (VP-16), doxorubicin, ifosphophamide, cyclophosphamide, etc.

Anti-prostaglandins include, but are not limited to, indomethacin, ketorolac tromethamine 0.5% (±)-5-benzoyl-2,3-dihydro-1H-pyrrolizine-1-carboxylic acid, compound with 2-amino-2-(hydroxymethyl)-1,3-propanediol (1:1) (ACULAR® Allegan, Irvine Calif.), OCUFEN® (flurbiprofen sodium 0.03%), meclofenamate, fluorbiprofen, and compounds in the pyrrolo-pyrrole group of non-steroidal anti-inflammatory drugs.

A matrix metalloproteinase inhibitor may be added. These include, but are not limited to, doxycycline, TIMP-1, TIMP-2, TIMP-3, TIMP-4, MMP1, MMP2, MMP3, Batimastat, or marimastat.

Anti-angiogenesis agents include, but are not limited to, antibodies to vascular endothelial growth factor (VEGF) such as bevacizumab (AVASTIN®) and rhuFAb V2 (ranibizumab) (Genentech), and other anti-VEGF compounds; pigment epithelium derived factor(s) (PEDF); CELEBREX®; VIOXX®; interferon alpha; interleukin-12 (IL-12); thalidomide and derivatives such as REVIMID™ (CC-5013) (Celgene Corporation); squalamine; endostatin; angiostatin; the ribozyme inhibitor ANGIOZYME® (Sima Therapeutics); multifunctional antiangiogenic agents such as NEOVASTAT® (AE-941) (Aeterna Laboratories, Quebec City, Canada); etc., as known to one skilled in the art.

Other agents may also be added, such as NSAIDS, vitamins, minerals, cytokines, growth factors, etc. Examples of the above include, but are not limited to, colchicine, naproxen sodium (ANAPROX® and ANAPROX DS®, (Roche); flurbiprofen (ANSAID®, Pharmacia Pfizer); diclofenac sodium and misoprostil (ARTHROTEC®, Searle Monsanto); valdecoxib (BEXTRA®, Pfizer); diclofenac potassium (CATAFLAM®), Novartis); celecoxib (CELEBREX®, Searle Monsanto); sulindac (CLINORIL®, Merck); oxaprozin (DAYPRO®), Pharmacia Pfizer); salsalate (DISALCID®, 3M); salicylate (DOLOBID®), Merck); naproxen sodium (EC NAPROSYN®, Roche); piroxicam (FELDENE®, Pfizer); indomethacin (INDOCIN®, Merck); etodolac (LODINE®), Wyeth); meloxicam (MOBIC®, Boehringer Ingelheim); ibuprofen (MOTRIN®, Pharmacia Pfizer); naproxen (NAPRELAN®, Elan); naproxen (NAPROSYN®, Roche); ketoprofen (ORUDIS®), ORUVAIL®, Wyeth); nabumetone (RELAFEN®, SmithKline); tolmetin sodium (TOLECTIN®, McNeil); choline magnesium trisalicylate (TRILISATE®, Purdue Fredrick); rofecoxib (VIOXX®, Merck), vitamins A, B (thiamine), B₆ (pyridoxine), B₁₂ (cobalamine), C (ascorbic acid), D₁, D₂ (ergocalciferol), D₃ (cholcalciferol), E, K (phytonadione), K₁ (phytylmenaquinone), K₂ (multiprenylmenaquinone); carotenoids such as lutein and zeaxanthin; macrominerals and trace minerals including, but not limited to, calcium, magnesium, iron, iodine, zinc, copper, chromium, selenium, manganese, molybdenum, fluoride, boron, etc. Commercially available supplements are also included such as high potency zinc (commercially available as OCUVITE® PRESERVISION®, Bausch & Lomb, Rochester N.Y.), or high potency antioxidants (zinc, lutein, zeaxanthin) (commercially available as ICAPS® Dietary Supplement, Alcon, Fort Worth Tex.).

In one example, rabbits are administered neurostimulatory or neuroprotective macrolides, macrolide analogs, neurotrophins, and/or neuropoietic factors (treated) or vehicle alone (control). To generate damage to retinal sensory or ganglion cells, anesthetized rabbits may be subjected to a sever crush injury of the optic nerve, or may be treated to induce increased intraocular pressure, or may be treated to induce retinal ischemic/reperfusion injury, or may be subject to other methods known to one skilled in the art.

As an example of one embodiment, after excising the conjunctiva and exposing the optic nerve with the aid of a binocular operating microscope with care not to interfere with the blood supply, the nerve can be mechanically crushed for a defined period using forceps or other instruments, as described in Schori et al., PNAS 98:3398(2001), which is expressly incorporated by reference herein in its entirety.

As an example of another embodiment, rabbits may be treated to result in intraocular pressure greater than 17 mm Hg. This may be done by negative pressure applied to a corneoscleral ring fixed to the sclera and connected to a vacuum source, as known to one skilled in the art. This may also be done by positive pressure applied through a cannula connected to the interior chamber. This may also be done by blocking aqueous outflow using 80-120 applications of blue-green argon laser as described in Bakalash et al., Investigative Ophthalmology & Visual Science 44: 3374 (2003), which is expressly incorporated by reference herein.

As an example of another embodiment, the central and choroidal arteries may be surgically closed. Deprivation of blood flow to the retina would result in ischemia due to lack of oxygen and nutrients, while reperfusion would result in free radical injury; this type of ischemia/reperfusion injury is known to one skilled in the art.

One or a combination of the macrolides, macrolide analogues, neurotrophins, and neuropoietic factors in different combinations of agent, dose, route of administration, intervals, etc. as described herein may be used and administered as previously described.

Assessment of retinal damage in control and treated animals may be by applying dextran tetramethylrhodamine, a hydrophilic neurotracer (Molecular Probes, Eugene Oreg.) into the intraorbital portion of the optic nerve, with only functional axons capable of dye uptake. Rabbits are sacrificed twenty-four hours after dye administration, retinas excised, wholemounted, and preserved in 4% paraformaldehyde. Retinal ganglial cells are counted under 800× magnification using a fluorescence microscope. Four fields from each retina are counted with the same diameter and located the same distance from the optic disc. Eyes from untreated rabbits are used as controls.

Other dyes or markers for viable ganglion cells can be introduced and the number of cells can be counted in treated groups versus control groups. In addition, factors other than dye uptake can be used as an indicator of neuroprotection and/or neurostimulation. These factors include retinal ganglial and/or sensory cell morphology from treated versus control groups, assays of cellular function, conductivity, etc. Conversely, apoptosis may be assayed in retinal ganglial and/or sensory cells from treated versus control groups. For example, Annexin V binding is known by one skilled in the art as an indirect indicator of apoptosis, and binding can be assayed in treated versus control cells. A clonagenic assay is known by one skilled in the art as a direct indicator of apoptosis, and can be performed with the results compared from both treated and control cells.

It will be appreciated that the agents include pharmaceutically acceptable salts and derivatives thereof (e.g., sodium, potassium, bicarbonate, sulfate, etc). It will also be appreciated that the above lists are representative only and are not exclusive. The indications, effective doses, formulations (including buffers, salts, and other excipients), contraindications, vendors, etc. of each of the above are known to one skilled in the art.

In one embodiment, the composition is formulated for topical application. In another embodiment, the composition is formulated for intraocular application. In another embodiment, the composition is formulated for subconjunctival or intravitreal application. In another embodiment, the composition is in a delayed- or extend-release formulation. In another embodiment, the composition is formulated on or in an intraocular lens (e.g., implanted lens, contact lens). In another embodiment, the composition is formulated on or in an implanted ocular device. None of these formulations result in significant systemic absorption, so that there are no detrimental effects that may result with systemically administered macrolides and/or neurostimulatory factor(s).

The formulation may be a slow, extended, or time release formulation, a carrier formulation such as microspheres, microcapsules, liposomes, etc., as known to one skilled in the art. Any of the above-mentioned delayed release delivery systems may be administered topically, intraocularly, subconjunctivally, or by implant to result in sustained release of the agent over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered topically or through a needle, or may be implanted. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, New York 2000), the relevant sections of which are incorporated by reference herein in their entirety. For example, a sustained release intraocular implant may be inserted through the pars plana for implantation in the vitreous cavity. An intraocular injection may be into the vitreous (intravitreal), or under the conjunctiva (subconjunctival), or behind the eye (retrobulbar), or under the Capsule of Tenon (sub-Tenon), and may be in a depot form. The composition may be administered via a contact lens applied to the exterior surface of an eye, with the composition incorporated into the lens material (e.g., at manufacture, or contained in a lens solution). The composition may be administered via an intraocular lens (IOL) that is implanted in the eye. Implantable lenses include any IOL used to replace a patient's diseased lens following cataract surgery, including but not limited to those manufactured by Bausch and Lomb (Rochester N.Y.), Alcon (Fort Worth Tex.), Allergan (Irvine Calif.), and Advanced Medical Optics (Santa Ana Calif.). When the lens is implanted within the lens capsule, the composition provides the desired effect to the eye. Concentrations suitable for implants (lenses and other types) and by contact lens administration may vary, as will be appreciated by one skilled in the art. For example, an implant may be loaded with a high amount of agent, but formulated or regulated so that a required concentration within the above-described ranges is sustainedly released (e.g., slow release formulation).

In various embodiments, the composition is administered up to four times a day. In embodiments where the composition is administered after surgery, administration may commence following surgery on the same day, or the day after surgery, or a few days after surgery, or any time after surgery. The composition may be self-administered or administered by another, for example, if visual acuity is poor, or if the patient is uncomfortable with self-administration. The patient is periodically evaluated (e.g., daily, every other day, etc.) using assessment methods known to one skilled in the art. In embodiments where the composition is used to assess corneal sensation, these include assessment of corneal clarity, corneal sensation (e.g., using a Cochet-Bonnet filament-type aesthesiometer), corneal enervation, etc. In embodiments where the composition is used to enhance ocular neuroprotection and/or neurostimulation, these may include one or more of the following assessments: retinal ganglial cell viability, quantitation of ocular glutamate levels, visual field and visual acuity determinations, assessment of visual evoked potential (VEP) to evaluate visual neural pathways via electrode measurement of brain electrical activity while watching a moving pattern on a video monitor, electroretinogram (ERG) to evaluate the ocular electrical responses to a flash of light using an electrode placed on the surface of the eye (e.g., cornea), electrooculargram (EOG), critical flicker fusion (CFF) test that measures a sensitivity threshold to provide information about the temporal responsiveness of visual pathways, etc. These assessments are known to one skilled in the art.

Other variations or embodiments of the invention will also be apparent to one of ordinary skill in the art from the above description. As one example, the invention may be used to facilitate growth of transplanted neuronal cells, either mature or immature, and/or stem cells in the eye or brain. As another example, other ocular routes of administration and injection sites and forms are also contemplated. As another example, the invention may be used in patients who have experienced ocular trauma, ischemia, inflammation, etc. Thus, the forgoing embodiments are not to be construed as limiting the scope of this invention.

What is claimed is:

1. An ocular method comprising locally administering to an eye of an individual a biocompatible formulation of at least one neurostimulatory or neuroprotective macrolide or macrolide analog, optionally further including at least one neurotrophin or neuropoietic factor of the interleukin-6 receptor family, under conditions to result in enhanced ocular neuroprotection or neurostimulation.

2. The method of claim 1 wherein viability and/or activity of retinal sensory or ganglion cells is increased over viability and/or activity of the cells in the absence of the macrolide and/or macrolide analog.

3. The method of claim 1 wherein the formulation is administered prophylactically to a patient at risk for glaucoma or retinitis pigmentosa.

4. The method of claim 1 wherein the formulation is administered therapeutically to a patient with increased intraocular pressure, open angle glaucoma, and/or retinitis pigmentosa.

5. The method of claim 1 wherein the formulation is administered in conjunction with a current treatment for increased intraocular pressure to result in a synergistic effect.

6. The method of claim 5 wherein a macrolide or macrolide analog is administered in conjunction with acetazolamide.

7. The method of claim 1 wherein the formulation is administered as an extended release formulation.

8. The method of claim 1 wherein the formulation is administered topically, by intraocularly injection, by injection in the lens, on an ocular lens, or via an ocular device.

9. The method of claim 1 wherein the neurotrophin is at least one of nerve growth factor-β (NGFβ), brain-derived neurotrophic factor (BDNF), neurotrophin 3 (NT-3), neurotrophin 4 (NT-4), neurotrophin 6, ciliary neurotrophic factor (CNTF), or glial cell derived neurotrophic factor (GDNF), and the neuropoietic factor is at least one of leukemia inhibitory factor (LIF), ciliary neurotrophic factor (CNTF), oncostatin M, growth-promoting activity, or cardiotrophin 1.

10. The method of claim 1 wherein the macrolide is cyclosporin A.

11. The method of claim 1 wherein the macrolide is tacrolimus.

12. The method of claim 1 wherein the macrolide is sirolimus.

13. The method of claim 1 wherein the macrolide is everolimus.

14. The method of claim 1 wherein the macrolide is pimocrolous.

15. The method of claim 1 wherein the macrolide is at least one of erythromycin, azithromycin, clarithromycin, lincomycin, dirithromycin, josamycin, spiramycin, diacetyl-midecamycin, troleandomycin, tylosin, roxithromycin, ABT-773, telithromycin, macrolides derived from leucomycins, lincosamides, or derivatives thereof.

16. An ocular method comprising locally administering to an eye of an individual a biocompatible formulation of at least one neurostimulatory and/or neuroprotective macrolide or macrolide analog, optionally further including at least one neurotrophin or neuropoietic factor, under conditions to result in enhanced viability and/or activity of retinal sensory or ganglion cells over viability and/or activity of the cells in the absence of the macrolide or macrolide analog.

17. The method of claim 16 administered to a patient having or at risk for developing glaucoma.

18. The method of claim 16 administered to a patient having or at risk for developing retinitis pigmentosa.
